(19) United States
(12) Patent Application Publication
(10) Pub. No.: US 2023/0398099 A1
(43) Pub. Date: Dec. 14, 2023

(54) METHODS OF TREATING CONDITIONS RELATED TO THE S1P1 RECEPTOR

(71) Applicant: Arena Pharmaceuticals, Inc., New York, NY (US)

(72) Inventor: Amit D. MUNSHI, Park City, UT (US)

(21) Appl. No.: 18/250,560

(22) PCT Filed: Nov. 8, 2021
(86) PCT No.: PCT/US21/58488

§ 371 Date: Apr. 26, 2023

Related U.S. Application Data
(60) Provisional application No. 63/111,467, filed on Nov. 9, 2020.

(51) Int. Cl. A61K 31/4245; A61P 11/06; A61P 17/00

(52) U.S. Cl. CPC A61K 31/4245; A61P 11/06; A61P 17/00

(57) ABSTRACT

Provided are methods of treatment of a sphingosine 1-phosphate subtype 1 (S1P1) receptor-associated disorder comprising prescribing and/or administering to an individual in need thereof a standard dose of 1-{2-fluoro-4-[5-(4-isobutylpheny 1)-1,2,4-oxadiazole-3-yl]benzyl}-3-azetidinecarboxylic acid (Compound 1), or a pharmaceutically acceptable salt, hydrate, or solvate thereof.

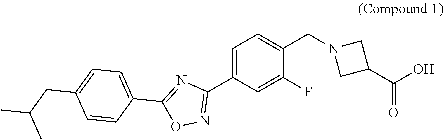 [SMILES: CC(C)Cc1ccc(-c2nc(-c3ccc(CN4CC(C(=O)O)C4)c(F)c3)no2)cc1]

Provided are methods useful in the treatment of sphingosine 1-phosphate subtype 1 (S1P₁ or SIP1) receptor-associated disorders, such atopic dermatitis and eosinophilic GI diseases, including eosinophilic esophagitis.

The sphingosine-1-phosphate (SIP) receptors 1-5 constitute a family of G protein-coupled receptors with a seven-transmembrane domain. These receptors, referred to as S1P₁ to S1P₅ (formerly termed endothelial differentiation gene (EDG) receptor-1, -5, -3, -6, and -8, respectively) are activated via binding by sphingosine-1-phosphate, which is produced by the sphingosine kinase-catalyzed phosphorylation of sphingosine. S1P₁, S1P₄, and S1P₅ receptors activate Gi but not Gq, whereas S1P₂ and S1P₃ receptors activate both Gi and Gq. The S1P₃ receptor, but not the S1P₁ receptor, responds to an agonist with an increase in intracellular calcium.

Atopic dermatitis, also known as atopic eczema, is a chronic inflammatory skin condition characterized by pruritic, erythematous, and scaly skin lesions often localized to the flexural surfaces of the body. It can present with asthma and allergic rhinitis as part of an allergic triad; an estimated 30 percent of children with atopic dermatitis develop asthma later in life. The onset of atopic dermatitis generally is before two years of age, with only 10 percent of cases diagnosed after five years of age.—A 2003 survey of children in the United States estimated an overall prevalence of approximately 11 percent, and as high as 19 percent in some states.—A 2007 U.S. population-based survey suggested an estimated 17.8 million persons are living with atopic dermatitis, and most cases have not been diagnosed.—Early diagnosis and treatment may prevent significant morbidity from sleep disturbances, chronic postinflammatory skin changes, scarring from picking and scratching, and the development of secondary skin infections with Staphylococcus, Streptococcus, and herpes species.

Chronic atopic dermatitis demonstrates lichenification from repeated scratching. See FIGS. 3 and 4. Atopic dermatitis tends to involve the flexural surfaces of the body, anterior and lateral neck, eyelids, forehead, face, wrists, dorsa of the feet, and hands.

Esophageal inflammation disorders such as eosinophilic esophagitis (EoE), a disease characterized by high levels of eosinophils in the esophagus, as well as basal zonal hyperplasia, is increasingly being diagnosed in children and adults. Many aspects of the disease remain unclear including its etiology, natural history, and optimal therapy. EoE affects all age groups but most frequently individuals between 20 and 50 years of age. Symptoms of EoE often mimic those of gastroesophageal reflux disease (GERD) and include vomiting, dysphagia, pain and food impaction. The disease is painful, leads to difficulty swallowing, and predisposes patients to other complications. EoE is often misdiagnosed for GERD, causing delay in adequate treatment for EoE patients.

The compound 1-{2-fluoro-4-[5-(4-isobutylphenyl)-1,2,4-oxadiazole-3-yl]benzyl}-3-azetidinecarboxylic acid (Compound 1), or a pharmaceutically acceptable salt, solvate, or hydrate thereof,

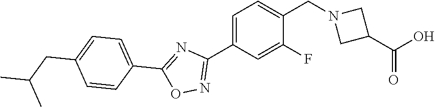 [SMILES: CC(C)Cc1ccc(-c2nc(-c3ccc(CN4CC(C(=O)O)C4)c(F)c3)no2)cc1]

has been reported as an S1P₁ agonist. See PCT patent publication WO2019/210511 and U.S. Pat. No. 10,280,158, hereby incorporated by reference in its entirety.

There exists a need for effectively treating individuals suffering from SIP1 receptor-associated disorders, such atopic dermatitis and an eosinophilic GI disease, as current therapies often provide only transient or marginal symptomatic relief. The present disclosure satisfies this need and provides related advantages as well.

Citation of any reference throughout this application is not to be construed as an admission that such reference is prior art to the present application.

### SUMMARY

Provided is a method of treating an individual with a SIP1 receptor-associated disorder, comprising: administering to the individual in need thereof a pharmaceutical dosage form comprising a therapeutically effective amount of 1-{2-fluoro-4-[5-(4-isobutylphenyl)-1,2,4-oxadiazole-3-yl]benzyl}-3-azetidinecarboxylic acid (Compound 1), or a pharmaceutically acceptable salt, solvate, or hydrate thereof.

Also provided is a method of treating an individual with atopic dermatitis, comprising: administering to the individual in need thereof a pharmaceutical dosage form comprising a therapeutically effective amount of 1-{2-fluoro-4-[5-(4-isobutylphenyl)-1,2,4-oxadiazole-3-yl]benzyl}-3-azetidinecarboxylic acid (Compound 1), or a pharmaceutically acceptable salt, solvate, or hydrate thereof.

Also provided is a method of treating an individual with an eosinophilic GI disease, comprising: administering to the individual in need thereof a pharmaceutical dosage form comprising a therapeutically effective amount of 1-{2-fluoro-4-[5-(4-isobutylphenyl)-1,2,4-oxadiazole-3-yl]benzyl}-3-azetidinecarboxylic acid (Compound 1), or a pharmaceutically acceptable salt, solvate, or hydrate thereof.

Also provided is a method of treating an individual with eosinophilic esophagitis (EoE), comprising: administering to the individual in need thereof a pharmaceutical dosage form comprising a therapeutically effective amount of 1-{2-fluoro-4-[5-(4-isobutylphenyl)-1,2,4-oxadiazole-3-yl]benzyl}-3-azetidinecarboxylic acid (Compound 1), or a pharmaceutically acceptable salt, solvate, or hydrate thereof.

Also provided is a method of treating an individual with asthma, comprising: administering to the individual in need thereof a pharmaceutical dosage form comprising a therapeutically effective amount of 1-{2-fluoro-4-[5-(4-isobutylphenyl)-1,2,4-oxadiazole-3-yl]benzyl}-3-azetidinecarboxylic acid (Compound 1), or a pharmaceutically acceptable salt, solvate, or hydrate thereof.

These and other aspects of the invention disclosed herein will be set forth in greater detail as the patent disclosure proceeds.

### BRIEF DESCRIPTION OF THE DRAWINGS

FIG. 1 depicts chronic atopic dermatitis in its lichenified form and associated hyperpigmentation.

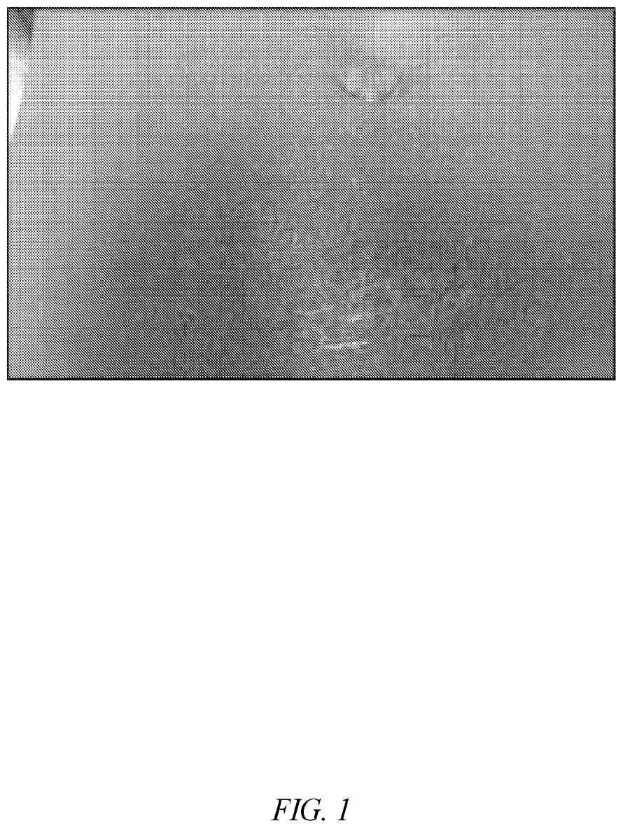

FIG. 2 depicts chronic atopic dermatitis demonstrating a lichenified plaque, as well as depigmentation resulting from repeated scratching.

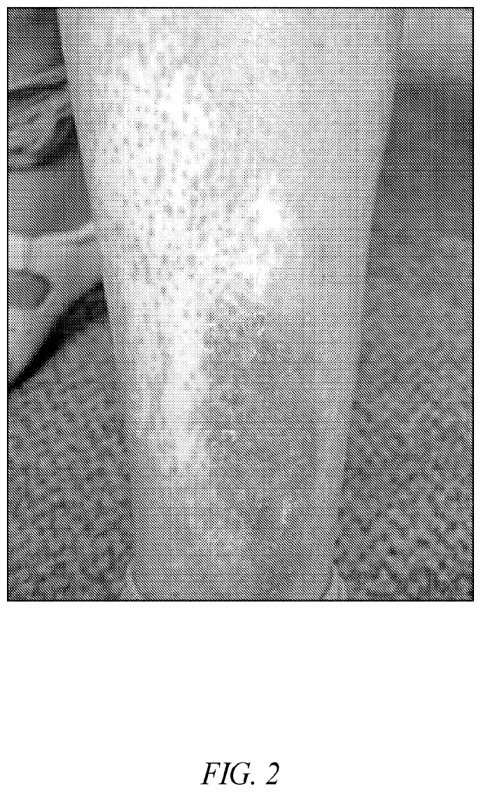

FIG. 3 depicts the pathogenesis of EoE. In step 1, esophageal epithelial cells polarize dendritic cells to a Th2 phenotype. In step 2, dendritic cells migrate to a lymph node (LN) and promote Th2 T cell differentiation. In step 3, newly activated Th2 cells leave the LN. In step 4, Th2 cells migrate to the esophagus and secrete cytokines. In step 5, eosinophils are recruited to the esophagus via the Th2 cytokines.

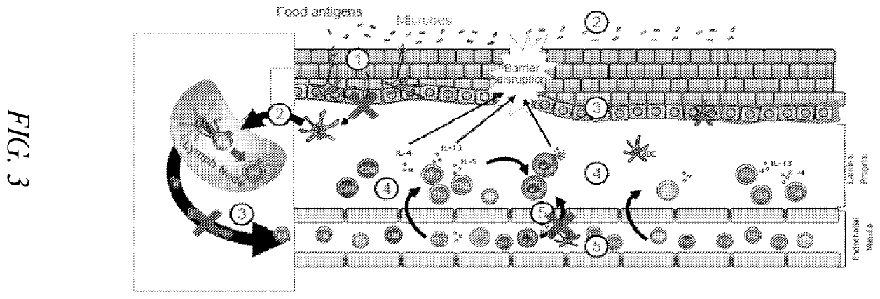

FIG. 4: Endoscopic manifestations in adult EoE patients enrolled in a European multicenter trial are shown: white exudate (a), longitudinal furrows (b), diffuse edema (c), fixed rings (d), severe stricture (e), and rings, furrows and edema (f).

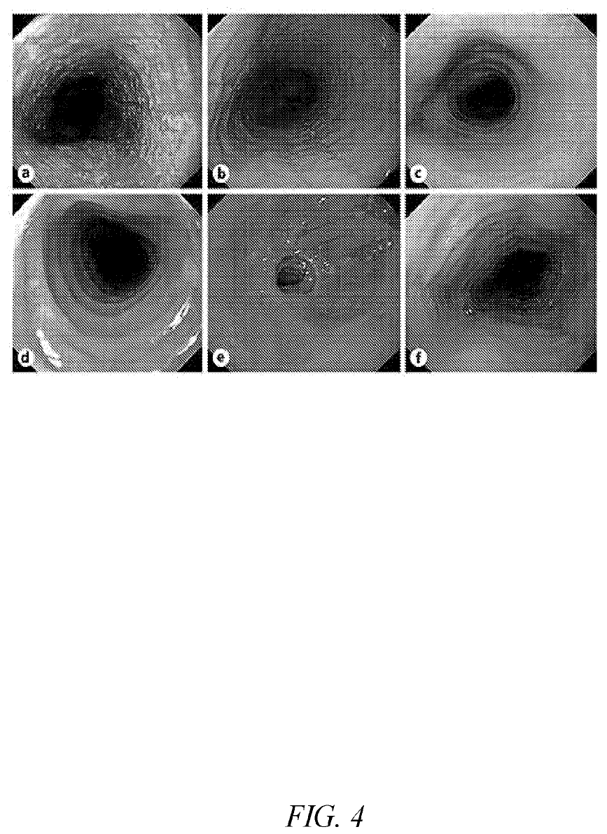

FIG. 5 depicts the schema for the study described in Example 6.

FIG. 6 shows the effect of Compound 1 (AR507630) on B and T cells in the skin.

FIG. 7 shows the effect of Compound 1 on white blood cell and lymphocyte frequency.

FIG. 8 shows the effect on Compound 1 on skin thickening.

FIG. 9 shows the effect of Compound 1 on immune cells in bronchoalveolar lavage (BAL).

FIG. 10 shows the effect on Compound 1 on lung function as measured by airway resistance or elastance.

### DETAILED DESCRIPTION

As used in the present specification, the following words and phrases are generally intended to have the meanings as set forth below, except to the extent that the context in which they are used indicates otherwise.

COMPOUND 1: As used herein, “Compound 1” means 1-{2-fluoro-4-[5-(4-isobutylphenyl)-1,2,4-oxadiazole-3-yl]benzyl}-3-azetidinecarboxylic acid including crystalline forms thereof. As a non-limiting example, Compound 1 may be any of form I, form IV, form XII, form II, form III, form V, form VI, form VII, form VIII, form IX, form X and form XI as described in CN105315266 (incorporated by reference herein in its entirety). As a non-limiting example, Compound 1 may be the sodium salt or crystalline form described in CN108299412 or WO2019/210511 (each of which is incorporated by reference herein in its entirety). Compound 1 may be prepared using techniques known in the art, including without limitation, the process described in CN105348276 (incorporated by reference herein in its entirety) or the process described in U.S. Pat. No. 10,280,158 (incorporated by reference herein in its entirety).

MODERATE TO SEVERE ATOPIC DERMATITIS: As used herein, “moderate to severe atopic dermatitis” means that 1 or more of the following features are present: (1) a minimum involvement of 10% body surface area (BSA); (2) regardless of BSA, individual lesions with moderate-to severe-features; involvement of highly visible areas or those important for function (e.g., neck, face, genitals, palms, and/or soles); and significantly impaired quality of life.

EXTRINSIC OR ALLERGIC ATOPIC DERMATITIS: Extrinsic or allergic atopic dermatitis is atopic dermatitis with high total serum IgE levels and the presence of specific IgE for environmental and food allergens.

INTRINSIC OR NON-ALLERGIC ATOPIC DERMATITIS: Intrinsic or non-allergic atopic dermatitis is atopic dermatitis with normal total IgE values and the absence of specific IgE.

EOSINOPHILIC ESOPHAGITIS: As used herein, “eosinophilic esophagitis” or “EoE” means an inflammatory disease characterized by abnormal eosinophilic inflammation within the esophagus and esophageal dysfunction. The primary symptoms of EoE include, but are not limited to, chest and abdominal pain, dysphagia, heartburn, food refusal, vomiting and food impaction. The clinicopathology of EoE is characterized by presence of ridges or trachea-like rings in the esophageal wall and eosinophilic infiltration in the esophageal mucosa. EoE is presently diagnosed by endoscopy of the esophagus followed by microscopic and biochemical analysis of the esophageal mucosal lining. EoE may be classified as allergic or non-allergic depending upon the status of the subject. The present invention includes methods to treat both allergic and non-allergic forms of EoE.

ESOPHAGEAL STRICTURES: As used herein, esophageal strictures can be classified as simple or complex, based on their diameter and associated anatomic abnormalities. A simple stricture is defined as a short stricture with a symmetric or concentric lumen and a diameter of ≥12 mm that can be traversed easily with an endoscope. A complex stricture is usually longer than 2 cm, may be angulated or irregular, and has a diameter of <12 mm. It may be associated with a large hiatal hernia, esophageal diverticula, or tracheoesophageal fistula. Complex strictures have a higher rate of recurrence and an increased risk for dilation-related adverse events, compared with simple strictures. The severity of a stricture can be estimated by the resistance encountered with passage of the diagnostic endoscope, which has a typical external diameter of 9 mm. A mild stricture allows passage of the endoscope without resistance, a moderate stricture offers increased resistance, whereas a severe stricture may not be traversable.

ALLERGEN: As used herein, “allergen,” means any substance, chemical, particle or composition which is capable of stimulating an allergic response in a susceptible individual. Allergens may be contained within or derived from a food item such as, e.g., dairy products (e.g., cow's milk), egg, wheat, soy, corn, rye, fish, shellfish, peanuts and tree nuts. Alternatively, an allergen may be contained within or derived from a non-food item such as, e.g., dust (e.g., containing dust mite), pollen, insect venom (e.g., venom of bees, wasps, mosquitoes, etc.), mold, animal dander, latex, medication, drugs, ragweed, grass and birch.

ALLERGIC RESPONSE or ALLERGIC REACTION or ALLERGIC SYMPTOM: As used herein, the phrases “allergic response,” “allergic reaction,” “allergic symptom,” and the like, include one or more signs or symptoms selected from urticaria (e.g., hives), angioedema, rhinitis, asthma, vomiting, sneezing, runny nose, sinus inflammation, watery eyes, wheezing, bronchospasm, reduced peak expiratory flow (PEF), gastrointestinal distress, flushing, swollen lips, swollen tongue, reduced blood pressure, anaphylaxis, and organ dysfunction/failure. An “allergic response,” “allergic reaction,” “allergic symptom,” etc., also includes immunological responses and reactions such as, e.g., increased IgE production, increased allergen-specific immunoglobulin production and/or eosinophilia.

EOSINOPHILIC INFILTRATION: As used herein, “eosinophilic infiltration” refers to the presence of eosinophils in an organ or tissue including blood, esophagus, stomach, duodenum, and ileum of a subject and more specifically, to presence of eosinophils in the mucosal lining of a region of the gastro-intestinal tract including, but not limited to, esophagus and stomach. Eosinophilic infiltration is analyzed, for example, in an esophageal tissue biopsy of a subject suffering from EoE. According to some embodiments, “eosinophilic infiltration” refers to the presence of ≥15 eosinophils per high power field in the esophagus. The term “high power field” refers to a standard total magnification of 400× by a microscope used to view eosinophils in a tissue, e.g., from the esophagus of a subject. In certain embodiments, “eosinophilic infiltration” includes infiltration into a tissue by leucocytes, for example, lymphocytes, neutrophils and mast cells. The leucocyte infiltration into, e.g., esophageal tissue can be detected by cell surface markers such as eosinophil-specific markers (e.g., CD11c^{Low/Neg}, SiglecF⁺, F4/80⁺, EMR1⁺, Siglec 8⁺, and MBP2⁺), macrophage-specific markers (e.g., CD11b⁺, F4/80⁺, CD14⁺, EMR1⁺, and CD68⁺), neutrophil-specific markers (e.g., CD11b⁺, Ly6G⁺, Ly6C⁺, CD11b⁺, and CD66b⁺), and T-cell-specific markers (e.g., CD3⁺ CD4⁺ CD8⁺).

REDUCTION IN ESOPHAGUS EOSINOPHILS: As used herein, “a reduction in esophagus eosinophils” means that the number of eosinophils and other leucocytes measured in the esophagus of a subject with EoE and who has been treated with Compound 1, or a pharmaceutically acceptable salt or as a solvate or hydrate thereof, is at least 5%, 10%, 20%, 50%, 70%, 80%, or 90% lower than the esophagus eosinophils measured in the same or an equivalent subject that has not been treated with Compound 1, or a pharmaceutically acceptable salt or as a solvate or hydrate thereof. In certain embodiments, reducing eosinophilic infiltration means detecting less than 15 eosinophils per high power field, such as less than 10 eosinophils, less than 9 eosinophils, less than 8 eosinophils, less than 7 eosinophils, less than 6 eosinophils, or less than 5 eosinophils per high power field in a biopsy of the esophageal mucosa. In certain embodiments, a reduction in esophagus eosinophils means that no eosinophils are detected in the esophageal mucosa of a subject.

EOE-ASSOCIATED BIOMARKER: As used herein, the term “EoE-associated biomarker” means any biological response, cell type, parameter, protein, polypeptide, enzyme, enzyme activity, metabolite, nucleic acid, carbohydrate, or other biomolecule which is present or detectable in an EoE patient at a level or amount that is different from (e.g., greater than or less than) the level or amount of the marker present or detectable in a non-EoE patient. Exemplary EoE-associated biomarkers include, but are not limited to, e.g., esophagus eosinophils, eotaxin-3 (CCL26), periostin, serum IgE (total and allergen-specific), IL-13, IL-5, serum thymus and activation regulated chemokine (TARC; CCL17), thymic stromal lymphopoietin (TSLP), serum eosinophilic cationic protein (ECP), and eosinophil-derived neurotoxin (EDN). The term “EoE-associated biomarker” also includes a gene or gene probe known in the art which is differentially expressed in a subject with EoE as compared to a subject without EoE. For example, genes which are significantly up-regulated in a subject with EoE include, but are not limited to, T-helper 2 (Th2)-associated chemokines such as CCL8, CCL23 and CCL26, periostin, cadherin-like-26, and TNFα-induced protein 6. Alternatively, “EoE-associated biomarker” also includes genes which are downregulated due to EoE such as terminal differentiation proteins (e.g., filaggrin). Certain embodiments relate to use of these biomarkers for monitoring disease reversal with the administration of Compound 1, or a pharmaceutically acceptable salt or as a solvate or hydrate thereof. Methods for detecting and/or quantifying such EoE-associated biomarkers are known in the art; kits for measuring such EoE-associated biomarkers are available from various commercial sources; and various commercial diagnostic laboratories offer services which provide measurements of such biomarkers as well.

ADMINISTERING: As used herein, “administering” means to provide a compound or other therapy, remedy, or treatment such that an individual internalizes a compound.

CO-ADMINISTER: As used herein, “co-administer” and “co-administration” and variants thereof mean the administration of at least two drugs to a patient either subsequently, simultaneously, or consequently proximate in time to one another (e.g., within the same day, or week or period of 30 days, or sufficiently proximate that each of the at least two drugs can be simultaneously detected in the blood plasma). When co-administered, two or more active agents can be co-formulated as part of the same composition or administered as separate formulations. This also may be referred to herein as “concomitant” administration or variants thereof.

PRESCRIBING: As used herein, “prescribing” means to order, authorize, or recommend the use of a drug or other therapy, remedy, or treatment. In some embodiments, a health care practitioner can orally advise, recommend, or authorize the use of a compound, dosage regimen or other treatment to an individual. In this case the health care practitioner may or may not provide a prescription for the compound, dosage regimen, or treatment. Further, the health care practitioner may or may not provide the recommended compound or treatment. For example, the health care practitioner can advise the individual where to obtain the compound without providing the compound. In some embodiments, a health care practitioner can provide a prescription for the compound, dosage regimen, or treatment to the individual. For example, a health care practitioner can give a written or oral prescription to an individual. A prescription can be written on paper or on electronic media such as a computer file, for example, on a hand-held computer device. For example, a health care practitioner can transform a piece of paper or electronic media with a prescription for a compound, dosage regimen, or treatment. In addition, a prescription can be called in (oral), faxed in (written), or submitted electronically via the interne to a pharmacy or a dispensary. In some embodiments, a sample of the compound or treatment can be given to the individual. As used herein, giving a sample of a compound constitutes an implicit prescription for the compound. Different health care systems around the world use different methods for prescribing and/or administering compounds or treatments and these methods are encompassed by the disclosure.

A prescription can include, for example, an individual's name and/or identifying information such as date of birth. In addition, for example, a prescription can include: the medication name, medication strength, dose, frequency of administration, route of administration, number or amount to be dispensed, number of refills, physician name, physician signature, and the like. Further, for example, a prescription can include a DEA number and/or state number.

A healthcare practitioner can include, for example, a physician, nurse, nurse practitioner, or other related health care professional who can prescribe or administer compounds (drugs) for the treatment of a sphingosine 1-phosphate subtype 1 (S1P₁) receptor-associated disorder. In addition, a healthcare practitioner can include anyone who can recommend, prescribe, administer, or prevent an individual from receiving a compound or drug including, for example, an insurance provider.

PREVENT, PREVENTING, OR PREVENTION: As used herein, the term “prevent,” “preventing”, or “prevention” such as prevention of a sphingosine 1-phosphate subtype 1 (S1P₁) receptor-associated disorder or the occurrence or onset of one or more symptoms associated with the particular disorder and does not necessarily mean the complete prevention of the disorder. For example, the term “prevent,” “preventing” and “prevention” means the administration of therapy on a prophylactic or preventative basis to an individual who may ultimately manifest at least one symptom of a disease or condition but who has not yet done so. Such individuals can be identified on the basis of risk factors that are known to correlate with the subsequent occurrence of the disease. Alternatively, prevention therapy can be administered without prior identification of a risk factor, as a prophylactic measure. Delaying the onset of at least one symptom can also be considered prevention or prophylaxis.

TREAT, TREATING, OR TREATMENT: As used herein the term “treat,” “treating”, or “treatment” means the administration of therapy to an individual who already manifests at least one symptom of a disease or condition or who has previously manifested at least one symptom of a disease or condition. For example, “treating” can include alleviating, abating or ameliorating a disease or condition symptoms, preventing additional symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. For example, the term “treating” in reference to a disorder means a reduction in severity of one or more symptoms associated with that particular disorder. Therefore, treating a disorder does not necessarily mean a reduction in severity of all symptoms associated with a disorder and does not necessarily mean a complete reduction in the severity of one or more symptoms associated with a disorder.

TOLERATE: As used herein, an individual is said to “tolerate” a dose of a compound if administration of that dose to that individual does not result in an unacceptable adverse event or an unacceptable combination of adverse events. One of skill in the art will appreciate that tolerance is a subjective measure and that what may be tolerable to one individual may not be tolerable to a different individual. For example, one individual may not be able to tolerate headache, whereas a second individual may find headache tolerable but is not able to tolerate vomiting, whereas for a third individual, either headache alone or vomiting alone is tolerable, but the individual is not able to tolerate the combination of headache and vomiting, even if the severity of each is less than when experienced alone.

ADVERSE EVENT: As used herein, an “adverse event” is an untoward medical occurrence that is associated with treatment with Compound 1 or a pharmaceutically acceptable salt, solvate, or hydrate thereof.

IN NEED OF TREATMENT and IN NEED THEREOF: As used herein, “in need of treatment” and “in need thereof” when referring to treatment are used interchangeably to mean a judgment made by a caregiver (e.g., physician, nurse, nurse practitioner, etc. in the case of humans; veterinarian in the case of animals, including non-human mammals) that an individual or animal requires or will benefit from treatment. This judgment is made based on a variety of factors that are in the realm of a caregiver's expertise, but that includes the knowledge that the individual or animal is ill, or will become ill, as the result of a disease, condition or disorder that is treatable by the compounds of the invention. Accordingly, the compounds of the invention can be used in a protective or preventive manner; or compounds of the invention can be used to alleviate, inhibit or ameliorate the disease, condition or disorder.

INDIVIDUAL: As used herein, “individual” means any animal, including mammals, preferably mice, rats, other rodents, rabbits, dogs, cats, swine, cattle, sheep, horses, or primates and most preferably humans. In some embodiments, a human individual is referred to a “patient.”

DOSE: As used herein, “dose” means a quantity of Compound 1, or a pharmaceutically acceptable salt, solvate, or hydrate thereof, given to the individual for treating or preventing the disease or disorder at one specific time.

THERAPEUTICALLY EFFECTIVE AMOUNT: As used herein, “therapeutically effective amount” of an agent, compound, drug, composition or combination is an amount which is nontoxic and effective for producing some desired therapeutic effect upon administration to a subject or patient (e.g., a human subject or patient). The precise therapeutically effective amount for a subject may depend upon, e.g., the subject's size and health, the nature and extent of the condition, the therapeutics or combination of therapeutics selected for administration, and other variables known to those of skill in the art. The effective amount for a given situation is determined by routine experimentation and is within the judgment of the clinician. In some embodiments, the therapeutically effective amount is the standard dose.

PHARMACEUTICAL COMPOSITION: As used here, “pharmaceutical composition” means a composition comprising at least one active ingredient, such as Compound 1; including but not limited to, salts, solvates, and hydrates of Compound 1, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

AGONIST: As used herein, “agonist” means a moiety that interacts with and activates a G-protein-coupled receptor, such as the S1P₁ receptor, such as can thereby initiate a physiological or pharmacological response characteristic of that receptor. For example, an agonist activates an intracellular response upon binding to the receptor, or enhances GTP binding to a membrane. In certain embodiments, an agonist of the invention is an S1P₁ receptor agonist that is capable of facilitating sustained S1P₁ receptor internalization (see e.g., Matloubian et al., Nature, 427, 355, 2004).

ANTAGONIST: As used herein, “antagonist” means a moiety that competitively binds to the receptor at the same site as an agonist (for example, the endogenous ligand), but which does not activate the intracellular response initiated by the active form of the receptor and can thereby inhibit the intracellular responses by an agonist or partial agonist. An antagonist does not diminish the baseline intracellular response in the absence of an agonist or partial agonist.

INVERSE AGONIST: As used herein, “inverse agonist” means a moiety that binds to the endogenous form of the receptor or to the constitutively activated form of the receptor and which inhibits the baseline intracellular response initiated by the active form of the receptor below the normal base level of activity which is observed in the absence of an agonist or partial agonist, or decreases GTP binding to a membrane. In some embodiments, the baseline intracellular response is inhibited in the presence of the inverse agonist by at least 30%. In some embodiments, the baseline intracellular response is inhibited in the presence of the inverse agonist by at least 50%. In some embodiments, the baseline intracellular response is inhibited in the presence of the inverse agonist by at least 75%, as compared with the baseline response in the absence of the inverse agonist.

HYDRATE: As used herein, “hydrate” means a compound of the invention or a salt thereof, that further includes a stoichiometric or non-stoichiometric amount of water bound by non-covalent intermolecular forces.

SOLVATE: As used herein, “solvate” means a compound of the invention or a salt, thereof, that further includes a stoichiometric or non-stoichiometric amount of a solvent bound by non-covalent intermolecular forces. Preferred solvents are volatile, non-toxic, and/or acceptable for administration to humans in trace amounts.

The compounds according to the invention may optionally exist as pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfiric, tartaric, oxalic, p-toluenesulfonic and the like, such as those pharmaceutically acceptable salts listed by Berge et al., Journal of Pharmaceutical Sciences, 66:1-19 (1977), incorporated herein by reference in its entirety.

The acid addition salts may be obtained as the direct products of compound synthesis. In the alternative, the free base may be dissolved in a suitable solvent containing the appropriate acid and the salt isolated by evaporating the solvent or otherwise separating the salt and solvent. The compounds of this invention may form solvates with standard low molecular weight solvents using methods known to the skilled artisan.

It is understood that when the phrase “pharmaceutically acceptable salts, solvates and hydrates” or the phrase “pharmaceutically acceptable salt, solvate, or hydrate” is used when referring to Compound 1, it embraces pharmaceutically acceptable solvates and/or hydrates of Compound 1, pharmaceutically acceptable salts of Compound 1, as well as pharmaceutically acceptable solvates and/or hydrates of pharmaceutically acceptable salts of Compound 1. It is also understood that when the phrase “pharmaceutically acceptable solvates and hydrates” or the phrase “pharmaceutically acceptable solvate or hydrate” is used when referring to Compound 1 that are salts, it embraces pharmaceutically acceptable solvates and/or hydrates of such salts.

It will be apparent to those skilled in the art that the dosage forms described herein may comprise, as the active component, either Compound 1 or a pharmaceutically acceptable salt or as a solvate or hydrate thereof. Moreover, various hydrates and solvates of Compound 1 and their salts will find use as intermediates in the manufacture of pharmaceutical compositions. Typical procedures for making and identifying suitable hydrates and solvates, outside those mentioned herein, are well known to those in the art; see for example, pages 202-209 of K. J. Guillory, “Generation of Polymorphs, Hydrates, Solvates, and Amorphous Solids,” in: Polymorphism in Pharmaceutical Solids, ed. Harry G. Britain, Vol. 95, Marcel Dekker, Inc., New York, 1999. Accordingly, one aspect of the present disclosure pertains to methods of prescribing and/or administering hydrates and solvates of Compound 1 and/or its pharmaceutical acceptable salts, that can be isolated and characterized by methods known in the art, such as, thermogravimetric analysis (TGA), TGA-mass spectroscopy, TGA-Infrared spectroscopy, powder X-ray diffraction (XRPD), Karl Fisher titration, high resolution X-ray diffraction, and the like. There are several commercial entities that provide quick and efficient services for identifying solvates and hydrates on a routine basis. Example companies offering these services include Wilmington PharmaTech (Wilmington, DE), Avantium Technologies (Amsterdam) and Aptuit (Greenwich, CT).

The present disclosure includes all isotopes of atoms occurring in the present compounds, salts, solvates, and hydrates. Isotopes include those atoms having the same atomic number but different mass numbers. One aspect of the present invention includes every combination of one or more atoms in the present compounds, salts, solvates, and hydrates that is replaced with an atom having the same atomic number but a different mass number. One such example is the replacement of an atom that is the most naturally abundant isotope, such as ¹H or ¹²C, found in one the present compounds, salts, solvates, and hydrates, with a different atom that is not the most naturally abundant isotope, such as ²H or ³H (replacing ¹H), or ¹¹C, ^{—}C, or ¹⁴C (replacing ¹²C). When such a replacement has taken place it is commonly referred to as being isotopically-labeled. Isotopic-labeling of the present compounds, salts, solvates, and hydrates can be accomplished using any one of a variety of different synthetic methods know to those of ordinary skill in the art and they are readily credited with understanding the synthetic methods and available reagents needed to conduct such isotopic-labeling. By way of general example, and without limitation, isotopes of hydrogen include ²H (deuterium) and ³H (tritium). Isotopes of carbon include ¹¹C, ¹³C, and ¹⁴C. Isotopes of nitrogen include ¹³N and ¹⁵N. Isotopes of oxygen include ¹⁵O, ¹⁷O, and ¹⁸O. An isotope of fluorine includes ¹⁸F. An isotope of sulfur includes ³⁵S. An isotope of chlorine includes ³⁶Cl. Isotopes of bromine include ⁷⁵Br, ⁷⁶Br, ⁷⁷Br, and ⁸²Br. Isotopes of iodine include ¹²³I, ¹²⁴I, ¹²⁵I, and ¹³¹I. Another aspect of the present invention includes compositions, such as, those prepared during synthesis, preformulation, and the like, and pharmaceutical compositions, such as, those prepared with the intent of using in a mammal for the treatment of one or more of the disorders described herein, comprising one or more of the present compounds, salts, solvates, and hydrates, wherein the naturally occurring distribution of the isotopes in the composition is perturbed. Another aspect of the present invention includes compositions and pharmaceutical compositions comprising the compounds, salts, solvates, and hydrates, as described herein wherein the salt is enriched at one or more positions with an isotope other than the most naturally abundant isotope. Methods are readily available to measure such isotope perturbations or enrichments, such as, mass spectrometry, and for isotopes that are radio-isotopes additional methods are available, such as, radio-detectors used in connection with HPLC or GC.

Compounds of the present invention can be converted to “prodrugs.” The term “prodrugs” means compounds that have been modified with specific chemical groups known in the art and that when administered into an individual undergo biotransformation to give the parent compound. Prodrugs can thus be viewed as compounds of the invention containing one or more specialized non-toxic protective groups used in a transient manner to alter or to eliminate a property of the compound. In one general aspect, the “prodrug” approach is utilized to facilitate oral absorption. A thorough discussion is provided in T. Higuchi and V. Stella, Prodrugs as Novel Delivery Systems Vol. 14 of the A.C.S. Symposium Series; and in Bioreversible Carriers in Drug Design, ed. Edward B. Roche, American Pharmaceutical Association and Pergamon Press, 1987, both of which are hereby incorporated by reference in their entirety.

When an integer is used in a method disclosed herein, the term “about” can be inserted before the integer.

Throughout this specification, unless the context requires otherwise, the word “comprise”, or variations such as “comprises” or “comprising” will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps, or group of compositions of matter shall be taken to encompass one and a plurality (i.e., one or more) of those steps, compositions of matter, groups of steps, or groups of compositions of matter.

Each embodiment described herein is to be applied mutatis mutandis to each and every other embodiment unless specifically stated otherwise.

Those skilled in the art will appreciate that the invention(s) described herein is susceptible to variations and modifications other than those specifically described. It is to be understood that the invention(s) includes all such variations and modifications. The invention(s) also includes all of the steps, features, compositions and compounds referred to or indicated in this specification, individually or collectively, and any and all combinations or any two or more of said steps or features unless specifically stated otherwise.

The present invention(s) is not to be limited in scope by the specific embodiments described herein, which are intended for the purpose of exemplification only. Functionally-equivalent products, compositions, and methods are clearly within the scope of the invention(s), as described herein.

It is appreciated that certain features of the invention(s), which are, for clarity, described in the context of separate embodiments, can also be provided in combination in a single embodiment. Conversely, various features of the invention(s), which are, for brevity, described in the context of a single embodiment, can also be provided separately or in any suitable subcombination. For example, a method that recites prescribing and/or administering Compound 1 or a pharmaceutically acceptable salt, solvate, or hydrate thereof can be separated into two methods; one method reciting prescribing Compound 1 or a pharmaceutically acceptable salt, solvate, or hydrate thereof and the other method reciting administering Compound 1 or a pharmaceutically acceptable salt, solvate, or hydrate thereof. In addition, for example, a method that recites prescribing Compound 1 or a pharmaceutically acceptable salt, solvate, or hydrate thereof and a separate method of the invention reciting administering Compound 1 or a pharmaceutically acceptable salt, solvate, or hydrate thereof can be combined into a single method reciting prescribing and/or administering Compound 1 or a pharmaceutically acceptable salt, solvate, or hydrate thereof.

Provided is a method of treating an individual with a SIP1 receptor-associated disorder, comprising: administering to the individual in need thereof a pharmaceutical dosage form comprising a therapeutically effective amount of 1-{2-fluoro-4-[5-(4-isobutylphenyl)-1,2,4-oxadiazole-3-yl]benzyl}-3-azetidinecarboxylic acid (Compound 1), or a pharmaceutically acceptable salt, solvate, or hydrate thereof.

Also provided is a method of treating an individual with atopic dermatitis, comprising: administering to the individual in need thereof a pharmaceutical dosage form comprising a therapeutically effective amount of 1-{2-fluoro-4-[5-(4-isobutylphenyl)-1,2,4-oxadiazole-3-yl]benzyl}-3-azetidinecarboxylic acid (Compound 1), or a pharmaceutically acceptable salt, solvate, or hydrate thereof.

In some embodiments, the atopic dermatitis is chronic atopic dermatitis.

In some embodiments, the atopic dermatitis is moderate to severe atopic dermatitis.

In some embodiments, the individual was previously administered a topical therapy for the treatment of atopic dermatitis. In some embodiments, the individual had an inadequate response with, lost response to, or was intolerant to the topical therapy.

In some embodiments, the individual also is administered an emollient. In some embodiments, the emollient is chosen from collagen, elastin, glyceryl stearate and shea butter.

In some embodiments, the individual also is administered a therapeutic dose of a topical corticosteroid. In some embodiments, the topical corticosteroid is chosen from hydrocortisone, fluticasone, and betamethasone valerate.

In some embodiments, the individual also is administered a therapeutic dose of a topical calcineurin inhibitor. In some embodiments, the topical calcineurin inhibitor is chosen from tacrolimus and pimecrolimus.

In some embodiments, the individual also is administered ultraviolet phototherapy.

In some embodiments, the individual also is administered a therapeutic dose of an immunomodulatory agents. In some embodiments, the immunomodulatory agent is cyclosporine. In some embodiments, the immunomodulatory agent is interferon gamma-1b.

Also provided is a method of treating an individual with an eosinophilic GI disease, comprising: administering to the individual in need thereof a pharmaceutical dosage form comprising a therapeutically effective amount of 1-{2-fluoro-4-[5-(4-isobutylphenyl)-1,2,4-oxadiazole-3-yl]benzyl}-3-azetidinecarboxylic acid (Compound 1), or a pharmaceutically acceptable salt, solvate, or hydrate thereof.

Also provided is a method of treating an individual with eosinophilic esophagitis (EoE), comprising: administering to the individual in need thereof a pharmaceutical dosage form comprising a therapeutically effective amount of 1-{2-fluoro-4-[5-(4-isobutylphenyl)-1,2,4-oxadiazole-3-yl]benzyl}-3-azetidinecarboxylic acid (Compound 1), or a pharmaceutically acceptable salt, solvate, or hydrate thereof.

Also provided is a method of treating, preventing or ameliorating at least one symptom or indication of eosinophilic esophagitis (EoE) comprising: selecting an individual who exhibits at least one symptom or indication of EoE, wherein the individual has an elevated level of a biomarker selected from esophagus eosinophils, eotaxin-3, periostin, serum IgE (total and allergen-specific), IL-13, IL-5, TARC, TSLP, serum ECP, and EDN; and administering to the individual in need thereof a therapeutically effective amount of (R)-2-(7-(4-cyclopentyl-3-(trifluoromethyl)benzyloxy)-1,2,3,4-tetrahydrocyclopenta[b]indo1-3-yl)acetic acid (Compound 1), or a pharmaceutically acceptable salt, hydrate, or solvate thereof.

In some embodiments, the eosinophilic GI disease is selected from eosinophilic esophagitis (EoE), eosinophilic gastritis (EG), eosinophilic gastroenteritis (EGE), and eosinophilic colitis (EC).

In some embodiments, the individual is selected on the basis of exhibiting ≥15 eosinophils per high powered field (hpf) in the esophagus prior to or at the time of the treatment (“baseline”).

In some embodiments, the individual exhibits at least 50% decrease in the number of eosinophils per hpf from baseline at day 10 following the administration of Compound 1, or a pharmaceutically acceptable salt, hydrate, or solvate thereof.

In some embodiments, the individual is selected on the basis of exhibiting an eotaxin-3 level of greater than about 50 pg/mL prior to or at the time of initiation of treatment (“baseline”).

In some embodiments, the individual exhibits at least 50% decrease in eotaxin-3 level from baseline at day 10 following the administration.

Also provided is a method of treating, preventing or ameliorating at least one symptom or indication of eosinophilic esophagitis (EoE) comprising: selecting an individual having an allergic reaction to an allergen that renders the individual susceptible to EoE; and administering to the individual in need thereof a pharmaceutical dosage form comprising a therapeutically effective amount of (R)-2-(7-(4-cyclopentyl-3-(trifluoromethyl)benzyloxy)-1,2,3,4-tetrahydrocyclopenta[b]indo1-3-yl)acetic acid (Compound 1), or a pharmaceutically acceptable salt, hydrate, or solvate thereof.

In some embodiments, the individual is susceptible to an allergen. For example, the subject may exhibit one of the following characteristics: (a) is prone to allergic reactions or responses when exposed to one or more allergens; (b) has previously exhibited an allergic response or reaction to one or more allergens; (c) has a known history of allergies; and/or (d) exhibits a sign or symptom of an allergic response or anaphylaxis. In certain embodiments, the subject is allergic to an allergen associated with EoE or that renders the subject susceptible and/or prone to developing EoE.

In some embodiments, the individual exhibits an allergic reaction to a food allergen. For example, the subject may have an allergy to an allergen contained in a food item including, but not limited to, a dairy product, egg, wheat, soy, corn, rye, fish, shellfish, peanut, a tree nut, beef, chicken, oat, barley, pork, green beans, and fruits such as apple and pineapple.

In some embodiments, the individual is allergic to a non-food allergen such as allergens derived from dust, mold, insects, plants including pollen, and pets such as cats and dogs. Examples of non-food allergens (also known as environmental allergens or aeroallergens) include, but are not limited to, house dust mite allergens, pollen allergens, animal dander allergens, insect venom, grass allergens, and latex.

In some embodiments, the symptom or indication of EoE is selected from eosinophilic infiltration of the esophagus, thickening of the esophageal wall, food refusal, vomiting, abdominal pain, heartburn, regurgitation, dysphagia and food impaction.

In some embodiments, the individual, prior to treatment, exhibits (or have exhibited) one or more indications of EoE such as, e.g., esophageal overexpression of pro-inflammatory mediators such as mast cells, eosinophilic infiltration of the esophagus, thickening of the esophageal wall, dysphagia, food impaction and chest and abdominal pain and/or an elevated level of an EoE-associated biomarker.

In some embodiments, the individual is more susceptible to EoE or may show an elevated level of an EoE-associated biomarker. For example, the individual is suffering from an atopic disease or disorder such as food allergy, atopic dermatitis, asthma, allergic rhinitis and allergic conjunctivitis. In some embodiments, the subject has an inherited connective tissue disorder. Such a subject population may show an elevated level of an EoE-associated biomarker such as, e.g., IgE, eotaxin-3, periostin, IL-5, or IL-13.

In some embodiments, the individual shows elevated levels of one or more EoE-associated biomarkers. In some embodiments, the administration of Compound 1, or a pharmaceutically acceptable salt, hydrate, or solvate thereof, results in reducing the level of an EoE-associated biomarker in the individual. In some embodiments, the EoE-associated biomarker is selected from esophagus eosinophils, eotaxin-3, periostin, serum IgE (total and allergen-specific), IL-13, IL-5, serum thymus and activation regulated chemokine (TARC), thymic stromal lymphopoietin (TSLP), serum eosinophilic cationic protein (ECP), and eosinophil-derived neurotoxin (EDN).

In some embodiments, prior to treatment, the individual shows the presence of ≥15 eosinophils per high power field in the esophagus. In some embodiments, prior to treatment, the individual shows an elevated peripheral eosinophil counts (>300 cells/up or elevated serum IgE (>150 kU/L). In some embodiments, prior to treatment, the individual shows the presence of ≥15 eosinophils per high power field in the esophagus and an elevated peripheral eosinophil counts (>300 cells/up or elevated serum IgE (>150 kU/L).

In some embodiments, Compound 1, or a pharmaceutically acceptable salt, hydrate, or solvate thereof reduces migration of tissue dendritic cells to a lymph node.

In some embodiments, Compound 1, or a pharmaceutically acceptable salt, hydrate, or solvate thereof reduces infiltrating TH2 and CD8 T cells. In some embodiments, Compound 1, or a pharmaceutically acceptable salt, hydrate, or solvate thereof reduces circulating T cells.

In some embodiments, Compound 1, or a pharmaceutically acceptable salt, hydrate, or solvate thereof reduces tissue cytokines.

In some embodiments, Compound 1, or a pharmaceutically acceptable salt, hydrate, or solvate thereof reduces eosinophil infiltration.

In some embodiments, Compound 1, or a pharmaceutically acceptable salt, hydrate, or solvate thereof, reduces eosinophil tissue accumulation.

In some embodiments, the individual, prior to or at the time of administration of Compound 1, or a pharmaceutically acceptable salt, hydrate, or solvate thereof, has or is diagnosed with a disease or disorder selected from atopic dermatitis, asthma, allergic rhinitis and allergic conjunctivitis.

In some embodiments, the individual is, or was, treated with an IL-1 beta inhibitor. In some embodiments, the IL-1 beta inhibitor is anakinra, rilonacept, or canakinumab.

In some embodiments, the individual is, or was, treated with an IL-5 inhibitor. In some embodiments, the IL-5 inhibitor is benralizumab, mepolizumab or reslizumab.

In some embodiments, the individual is treated with an IL-9 inhibitor.

In some embodiments, the individual is, or was, treated with an IL-13 inhibitor. In some embodiments, the IL-13 inhibitor is lebrikizumab, RPC4046, or tralokinumab.

In some embodiments, the individual is, or was, treated with an IL-17 inhibitor. In some embodiments, the IL-17 inhibitor is ixekizumab or brodalumab.

In some embodiments, the individual is, or was, treated with an IL-25 inhibitor.

In some embodiments, the individual is, or was, treated with a TNFα inhibitor. In some embodiments, the TNFα inhibitor is SIMPONI® (golimumab), REMICADE® (infliximab), HUMIRA® (adalimumab), or CIMZIA® (certolizumab pegol).

In some embodiments, the individual is, or was, treated with an eotaxin-3 inhibitor.

In some embodiments, the individual is, or was, treated with an IgE inhibitor. In some embodiments, the IgE inhibitor is omalizumab.

In some embodiments, the individual is, or was, treated with a prostaglandin D2 inhibitor.

In some embodiments, the individual is, or was, treated with an immunosuppressant. In some embodiments, the immunosuppressant is AZASAN® (azathioprine), IMURAN® (azathioprine), GENGRAF® (cyclosporine), NEORAL® (cyclosporine), or SANDIMMUNE® (cyclosporine). Immunosuppressants also may be referred to as immunosuppressives or immunosuppressive agents.

In some embodiments, the individual is, or was, treated with a proton pump inhibitor. In some embodiments, the proton pump inhibitor is omeprazole, pantoprazole, esomeprazole, or dexlansoprazole.

In some embodiments, the individual is, or was, treated with a glucocorticoid. In some embodiments, the glucocorticoid is UCERIS® (budesonide); DELTASONE® (prednisone), MEDROL® (methylprednisolone), or hydrocortisone. Glucocorticosteroids also may be referred to as glucocorticoid or corticosteroids.

In some embodiments, the individual is, or was, treated with a NSAID. In some embodiments, the NSAID is aspirin, celecoxib, diclofenac, diflunisal, etodolac, ibuprofen, indomethacin, ketoprofen, ketorolac, nabumetone, naproxen, oxaprozin, piroxicam, salsalate, sulindac, or tolmetin.

In some embodiments, the individual is, or was, treated with allergen removal.

In some embodiments, the individual is, or was, treated with diet management. In some embodiments, diet management comprises targeted elimination diets wherein foods that test positive on allergy testing or history are removed from the diet.

In some embodiments, diet management comprises empiric six-food elimination. diet wherein instead of basing dietary elimination on allergy testing results, patients eliminate common allergy-causing foods (milk, eggs, wheat, soy, peanuts/tree nuts, fish/shellfish).

In some embodiments, diet management comprises an elemental diet wherein all sources of protein are removed from the diet and the patient drinks only an amino acid formula.

In some embodiments, diet management comprises food trial wherein specific foods are removed from the diet, and then added back, one at a time, to determine which food(s) cause a reaction. Diet management may involve repeat endoscopies with biopsies as foods are reintroduced to determine which foods are tolerated.

In some embodiments, prior to the treatment, the individual will not have severe strictures.

Also provided is a method of treating an individual with asthma, comprising: administering to the individual in need thereof a pharmaceutical dosage form comprising a therapeutically effective amount of 1-{2-fluoro-4-[5-(4-isobutylphenyl)-1,2,4-oxadiazole-3-yl]benzyl}-3-azetidinecarboxylic acid (Compound 1), or a pharmaceutically acceptable salt, solvate, or hydrate thereof.

In some embodiments, asthma is chosen from bronchial asthma, allergic asthma, intrinsic asthma, extrinsic asthma, dust asthma, chronic or inveterate asthma, late asthma and airway hyper-responsiveness.

In some embodiments, asthma is chosen from exercise-induced asthma, occupational asthma, and allergy-induced asthma.

In some embodiments, the pharmaceutical dosage form is administered once daily to the individual.

In some embodiments, the therapeutically effective amount is equivalent to about 0.1 mg to 2.5 mg of Compound 1. In some embodiments, the therapeutically effective amount is equivalent to, or to about, 0.1 mg to 5.0 mg of Compound 1. In some embodiments, the therapeutically effective amount is equivalent to, or to about, 0.1 mg, 0.2 mg, 0.25 mg, 0.3 mg, 0.4 mg, 0.5 mg, 0.6 mg, 0.7 mg, 0.75 mg 0.8 mg, 0.9 mg, 1.0 mg, 1.1 mg, 1.2 mg, 1.25 mg, 1.3 mg, 1.4 mg, 1.5 mg, 1.6 mg, 1.7 mg, 1.75 mg, 1.8 mg, 1.9 mg, 2.0 mg, 2.1 mg, 2.2 mg, 2.25 mg, 2.3 mg, 2.4 mg, 2.5 mg, 2.6 mg, 2.7 mg, 2.75 mg, 2.8 mg, 2.9 mg, 3.0 mg, 3.1 mg, 3.2 mg, 3.25 mg, 3.3 mg, 3.4 mg, 3.5 mg, 3.6 mg, 3.7 mg, 3.75 mg, 3.8 mg, 3.9 mg, 4.0 mg, 4.1 mg, 4.2 mg, 4.25 mg, 4.3 mg, 4.4 mg, 4.5 mg, 4.6 mg, 4.7 mg, 4.75 mg, 4.8 mg, 4.9 mg, or 5.0 mg of Compound 1. In some embodiments, the therapeutically effective amount is equivalent to about 0.15 mg of Compound 1. In some embodiments, the therapeutically effective amount is equivalent to about 0.25 mg of Compound 1. In some embodiments, the therapeutically effective amount is equivalent to about 0.5 mg of Compound 1. In some embodiments, the therapeutically effective amount is equivalent to about 1 mg of Compound 1. In some embodiments, the therapeutically effective amount is equivalent to about 1.5 mg of Compound 1. In some embodiments, the therapeutically effective amount is equivalent to about 2 mg of Compound 1. In some embodiments, the therapeutically effective amount is equivalent to about 2.5 mg of Compound 1. In some embodiments, the therapeutically effective amount is equivalent to about 3 mg of Compound 1. In some embodiments, the therapeutically effective amount is equivalent to about 4 mg of Compound 1. In some embodiments, the therapeutically effective amount is equivalent to about 5 mg of Compound 1.

In some embodiments, the Compound 1, or a pharmaceutically acceptable salt, hydrate, or solvate thereof, is administered orally.

In some embodiments, the individual has had an inadequate response with, lost response to, been intolerant to, or demonstrated dependence on another agent for the treatment of the SIP1 receptor-associated disorder. In some embodiments, the individual has had an inadequate response with the other agent for the treatment of the SIP1 receptor-associated disorder. In some embodiments, the individual has lost response to another agent for the treatment of the SIP1 receptor-associated disorder. In some embodiments, the individual was intolerant to another agent for the treatment of the SIP1 receptor-associated disorder.

In some embodiments, the individual has had an inadequate response with, lost response to, or been intolerant to a conventional therapy. In some embodiments, the individual has had an inadequate response to conventional therapy. In some embodiments, the individual has lost response to conventional therapy. In some embodiments, the prior conventional therapy is referred to as prior treatment.

In some embodiments, the Compound 1, or a pharmaceutically acceptable salt, hydrate, or solvate thereof, is formulated as a capsule or tablet suitable for oral administration.

Some embodiments of the present invention include a method of producing a pharmaceutical composition for “combination-therapy” comprising admixing at least one compound according to any of the compound embodiments disclosed herein, together with at least one known pharmaceutical agent as described herein and a pharmaceutically acceptable carrier.

Also provided are pharmaceutical compositions comprising a standard dose of Compound 1, or, a pharmaceutically acceptable salt, a hydrate or solvate thereof and, optionally, one or more pharmaceutically acceptable carriers. Also provided are pharmaceutical compositions comprising Compound 1, or, a pharmaceutically acceptable salt, a hydrate or solvate thereof, optionally, one or more pharmaceutically acceptable carriers. The carrier(s) must be “acceptable” in the sense of being compatible with the other ingredients of the formulation and not overly deleterious to the recipient thereof.

In some embodiments, Compound 1, or a pharmaceutically acceptable salt, a hydrate or solvate thereof, is administered as a raw or pure chemical, for example as a powder in capsule formulation.

In some embodiments, Compound 1, or a pharmaceutically acceptable salt, a hydrate or solvate thereof, is formulated as a pharmaceutical composition further comprising one or more pharmaceutically acceptable carriers.

Pharmaceutical compositions may be prepared by any suitable method, typically by uniformly mixing the active compound(s) with liquids or finely divided solid carriers, or both, in the required proportions and then, if necessary, forming the resulting mixture into a desired shape.

Conventional excipients, such as binding agents, fillers, acceptable wetting agents, tabletting lubricants and disintegrants may be used in tablets and capsules for oral administration. The compounds described herein can be formulated into pharmaceutical compositions using techniques well known to those in the art. Suitable pharmaceutically acceptable carriers, outside those mentioned herein, are known in the art; for example, see Remington, The Science and Practice of Pharmacy, 20^{th} Edition, 2000, Lippincott Williams & Wilkins, (Editors: Gennaro et al.)

For oral administration, the pharmaceutical composition may be in the form of, for example, a tablet or capsule. The pharmaceutical composition is preferably made in the form of a dosage unit containing a particular amount of the active ingredient. Examples of such dosage units are capsules, tablets, powders, granules or suspensions, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or encapsulating materials.

In powders, the carrier is a finely divided solid which is in a mixture with the finely divided active component.

In tablets, the active component is mixed with the carrier having the necessary binding capacity in suitable proportions and compacted to the desired shape and size.

The powders and tablets may contain varying percentage amounts of the active compound. A representative amount in a powder or tablet may be from 0.5 to about 90 percent of the active compound. However, an artisan would know when amounts outside of this range are necessary. Suitable carriers for powders and tablets include magnesium carbonate, magnesium stearate, talc, sugar, lactose, pectin, dextrin, starch, gelatin, tragacanth, methylcellulose, sodium carboxymethyl cellulose, a low melting wax, cocoa butter, and the like. The term “preparation” includes the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets or capsules. Also, the unit dosage form can be a capsule or tablet itself, or it can be the appropriate number of any of these in packaged form.

Further embodiments include the embodiments disclosed in the following Examples, which is not to be construed as limiting in any way.

### EXAMPLES

### Example 1

A randomized, double-blind, placebo-controlled, multiple dose study to evaluate the safety, tolerability, and efficacy of Compound 1 in atopic dermatitis patients who are inadequately controlled by or intolerant to topical therapy will be conducted. The primary outcome will be percent improvement from baseline in pruritus Visual Analogue Scale (VAS). Secondary outcome measures may include:
Improvement from baseline in Eczema Area and Severity Index (EASI)
Improvement from baseline in SCORing Atopic Dermatitis (SCORAD)
Improvement from baseline in static Investigator's Global Assessment (sIGA)
Improvement from baseline in Body Surface Area (BSA) of AD involvement Inclusion Criteria may include:
≥18 and ≥65 years of age at the time of consent.
Patients with Atopic Dermatitis
Pruritus visual analogue scale (VAS)≥50 mm at the screening and baseline visit
Eczema Area and Severity Index (EAST)≥10 at the screening and baseline visit
static Investigator's Global Assessment (sIGA) score≥3 at the baseline visit Exclusion Criteria may include:
Serological evidence of hepatitis B virus or hepatitis C virus infection
Known human immunodeficiency virus infection
Ongoing treatment with specific or non-specific hyposensitization therapy for AD
Treatment with mild or moderately potent topical corticosteroids (TCS) within 1 week prior to randomization
History of infection including skin infection requiring treatment with oral or intravenous (IV) antibiotics, antivirals, or antifungals within 1 week prior to randomization.
Evidence of tuberculosis (TB) infection as defined by a positive purified protein derivative (PPD) and/or positive interferon-gamma release assay.

### Example 2

Compound 1, or a pharmaceutically acceptable salt, solvate, or hydrate thereof, can be tested in an animal model of atopic dermatitis. After anesthetization, the dorsal skin of BALB/c mice is shaved and tape stripped to disrupt the skin barrier. 2,4-dinitrofluorobenzene (DNFB) is then applied on a 1×1 cm square of the dorsal skin of each mouse. DNFB or vehicle patch is removed 2 h later. The dorsal skin is painted once weekly for 5 weeks (i.e., five painting sessions). Scratching behavior of mice is recorded for 15 min. The record is performed 2 h after DNFB sensitization in the observation chamber using a digital video camera. Multiple scratching behavior around the rostral shaved area using hind paws is counted as one event.

Compound 1, or a pharmaceutically acceptable salt, solvate, or hydrate thereof, is applied to the skin of the mouse at a dose that is the human equivalent dose to 0.1 mg to 2.5 mg of Compound 1. See, e.g., FDA Guidance for Industry: Estimating the Maximum Safe Starting Dose in Initial Clinical Trials for Therapeutics in Adult Healthy Volunteers (2005). Scratching behavior is recorded for 15 minutes and compared to control.

### Example 3

Compound 1, or a pharmaceutically acceptable salt, solvate, or hydrate thereof, can be tested in an animal model of contact dermatitis using BALB/c mice. A FITC/Sham solution is applied to the shaved area of each flank on study days 0 and 5. A challenge occurs on study days 10, 11, and 12 by an application of the FITC/Sham solution on the dorsal surface of both ears. At study termination, one ear will be collected for flow cytometry.

The following groups of treatments will be tested:
1. Sham Control;
2. Disease Only Control;
3. Dexamethasone;
4. Vehicle Control;
5. Compound 1, or a pharmaceutically acceptable salt thereof at dose 1; and
6. Compound 1, or a pharmaceutically acceptable salt thereof at dose 2.
   Groups 4-6 will be dosed once daily by oral gavage starting on day 1 through day 12.

Readouts may include:
Ear thickness: Thickness of the right and left ears is measured via dial calipers at baseline and on days 0 and 5 post FITC sensitization and after days of challenge 10-12 and at termination on day 13. The average thickness of both the left and right ear will be determined.
Clinical signs: Careful clinical examinations are carried out daily. Observations include changes in skin, fur, eyes, mucous membranes, occurrence of secretions and excretions (e.g., diarrhea) and autonomic activity (e.g., lacrimation, salivation, piloerection, pupil size, unusual respiratory pattern). Changes in gait, posture and response to handling, as well as the presence of bizarre behavior, tremors, convulsions, sleep and coma are also noted.
Body weight: Mice body weight will be measured 3× weekly throughout the study.
FACS analysis: Reagents for flow cytometry will be ordered at the start of the study. FACS analysis will be performed on ear tissue samples harvested at study termination. The total cell count and cell differentiation will be carried out for CD45, CD3, TCRb, CD4, CD8, CD69, CD19, SiglecF as well as viability.
CBC differential: Blood samples collected at study termination will be assessed for complete blood count (CBC) differential.

### Example 4

6-8 week old female Balb/c mice will be received at least 3 days prior to study start. On Day 0 and Day 7, mice in Groups 2-5 will receive an intraperitoneal (IP) injection of OVA/Alum. On Days 13, 14, and 15, animals in Groups 2-5 will be administered 20 μg OVA by intranasal (IN) route. Animals will be dosed as shown in table below with compounds and sacrificed on at indicated time points. Study includes daily body weight and observations, survival, and blood (serum) and lung collection. Detailed lung mechanics using FlexiVent system will be performed following Methacholine challenge just prior to sacrifice. Left lung will be fixed in NBF for histopathology (H&E, PAS). Right lung will have BAL fluid collected for total and differential counts, and leftover BAL fluid will be frozen and stored at −80° C. and used for possible cytokine analysis (IL-5, IL-13, IL-4, IFNγ). Right lung will be flash frozen and stored at −80° C. for possible downstream analysis or until shipment to client.

| | OVA Sensitization | | | |
|---|---|---|---|---|
| | (IP) Day | | | Route & Dose |
| Group | 0 and Day 7 | Treatment | Dose | Schedule |
| 1 | — | — | — | — |
| 2 | 20 μg OVA + | Vehicle | — | PO, QD |
| 3 | 1.5 mg Alum | Compound 1 | Dose 1 | Day 12-15 |
| 4 | | | Dose 2 | |
| 5 | | Dexamethasone | 5 mg/kg | IP, QD |
| | | | | Day 12-15 |

### Example 5: In Vitro Receptor Pharmacology

Compound 1 was characterized in b-arrestin recruitment assays for human sphingosine-1-phosphate receptors 1-5 (hS1P₁₋₅) and GTPgS binding assays for hS1P₁. Reference S1P modulators (ozanimod, fingolimod(P), SIP) were also characterized as comparators. Summary tables for both b-arrestin recruitment and GTPgS binding assays are shown below.

b-Arrestin Recruitment Assays

| | | hS1PR₁ | hS1PR₂ | hS1PR₃ | hS1PR₄ | hS1PR₅ |
|---|---|---|---|---|---|---|
| Compound | EC₅₀^{a}, | 6.2 [2.9, | NR (4) | NR (4) | 3.2 [1.1, | 0.71 [0.34, |
| 1 | nM | 13.3] (4) | | | 8.9] (4) | 1.49] (5) |
| | Eₘₐₓ^{b} | 128 | NR (4) | NR (4) | 104 | 120 |
| Ozanimod | EC₅₀^{a}, | 20.9 [9.6, | NR (4) | NR (3) | 117 [52.7, | 7.3 [2.7, |
| | nM | 45.3] (4) | | | 259] (4) | 19.8] (5) |
| | Eₘₐₓ^{b} | 130 | NR (4) | NR (3) | 93 | 117 |
| Fingolimod(P) | EC₅₀^{a}, | 3.5 [2.2, | NR (4) | 10.2 [5.3, | 0.76 [0.17, | 1.8 [1.1, |
| | nM | 5.7] (4) | | 19.5] (4) | 3.38] (3) | 2.7] (3) |
| | Eₘₐₓ^{b} | 123 | NR (4) | 9 | 94 | 62 |
| S1P | EC₅₀^{a}, | 18.3 [6.7, | 17.3 [11.5, | 13.9 [6.7, | 0.99 [0.49, | 1.3 [0.7, |
| | nM | 47.8] (4) | 26.2] (3) | 29.1] (4) | 1.97] (4) | 2.2] (5) |
| | Eₘₐₓ^{b} | 113 | 108 | 113 | 117 | 114 |

^{a}EC₅₀ [95% confidence interval] (n).
^{b}Eₘₐₓ—% relative to S1P.
NR—No response

S1P2 Receptor Internalization

| | Compound 1 | Fingolimod(p) | S1P |
|---|---|---|---|
| Mean pEC₅₀ | 5.60 | 6.97 | 7.30 |
| Std Dev | 0.22 | 0.13 | 0.26 |
| Mean EC₅₀, nM | 2,512 | 107 | 50 |
| Efficacy, % of S1P | 65 | 80 | 100 |
| N | 5 | 5 | 5 |

NR—No Response.
NA—Not applicable.

S1P₁ GTPgS Binding Assays

| | | |
|---|---|---|
| Compound 1 | EC₅₀^{a}, nM | 1.8 [1.3, 2.5] (4) |
| | Eₘₐₓ^{b} | 98 |
| Ozanimod | EC₅₀^{a}, nM | 3.5 [2.2, 5.6] (4) |
| | Eₘₐₓ^{b} | 104 |
| Fingolimod(P) | EC₅₀^{a}, nM | 0.66 [0.36, 1.21] (3) |
| | Eₘₐₓ^{b} | 90 |
| S1P | EC₅₀^{a}, nM | 77 [57, 103] (4) |
| | Eₘₐₓ^{b} | 102 |

sf^{a} EC₅₀ [95% confidence interval] (n).
^{b}Eₘₐₓ—% relative to S1P.
NR—no response.

### Example 6: In Vivo Atopic Dermatitis Model

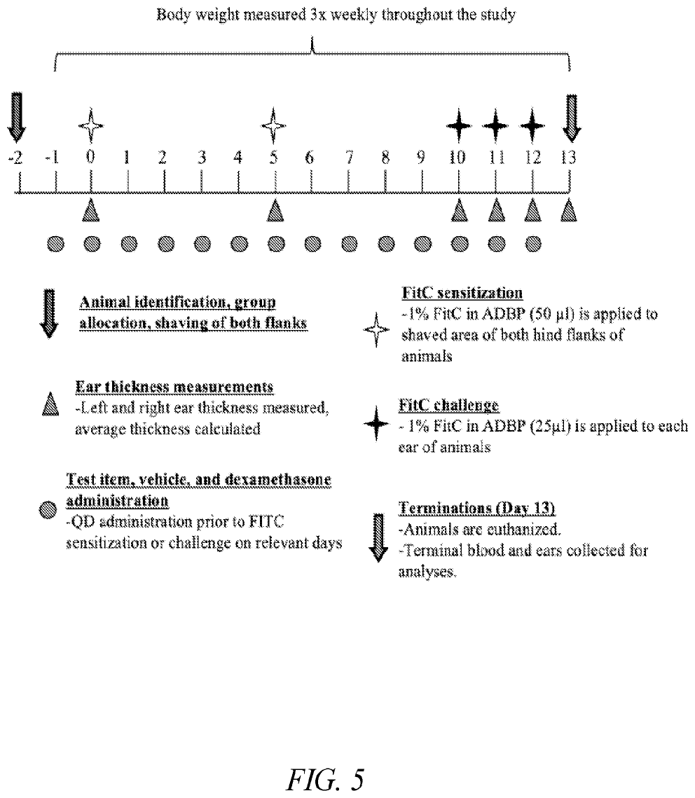

Compound 1 was tested in a mouse model of FITC-induced contact dermatitis. Fluorescein isothiocyanate isomer I (FITC) was used to induce contact hypersensitivity (CHS)/dermatitis in BALB/c mice by administering two sensitizing applications of the hapten onto the shaved flanks of the animals on Days 0 and 5, followed by three days of FITC challenge on the dorsal surface of both ears on Days 10-12. Animals were split into treatment groups that received daily oral administration of either 1 mg/kg or 0.1 mg/kg of the test article, Compound 1. Treatment was initiated on Day −1 to model prophylactic treatment. See FIG. 5.

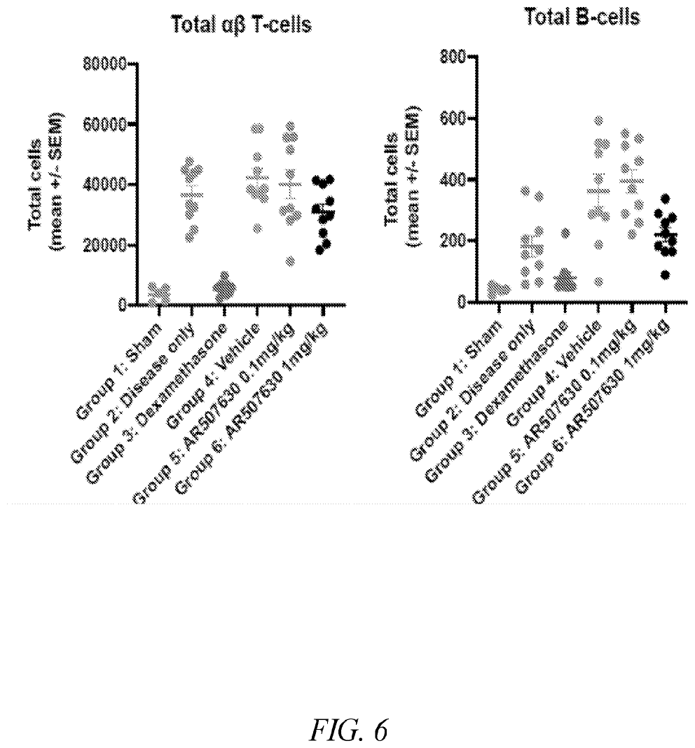

1 mg/kg Compound 1 reduced B cells and T cells in the skin (compared to vehicle). As expected from the reduction of circulating white blood cells (WBC) and lymphocytes, Compound 1 reduced T cells and B cells in the skin in a dose-dependent manner. See FIG. 6. Reduction of these cells corresponded with an improvement in disease (skin thickness).

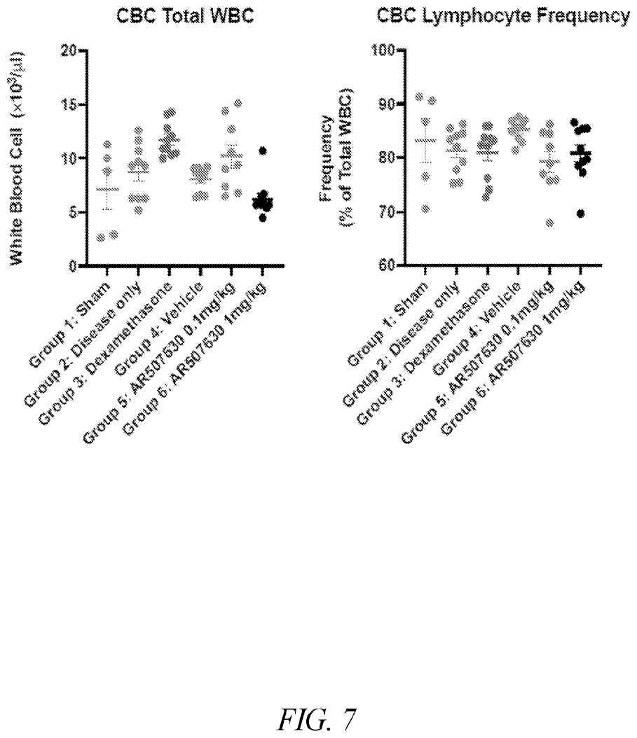

1 mg/kg Compound 1 reduced WBC count and lymphocyte frequency (compared to vehicle). Effects seen were dose-dependent. See FIG. 7.

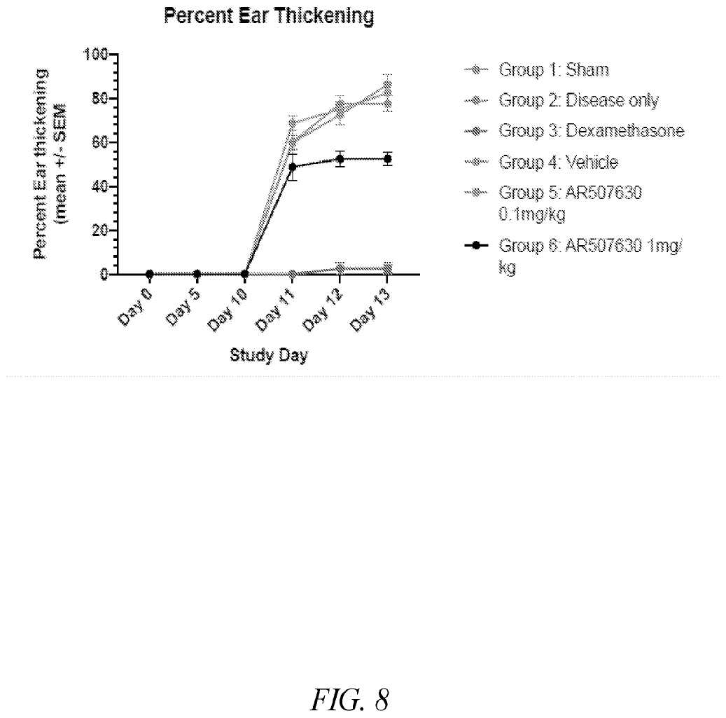

1 mg/kg Compound 1 reduced skin thickening (0.1 mg/kg had no effect). Ear thickening is a surrogate measure of gross inflammation, which is used as a measure of overall efficacy in this model. This data suggests Compound 1 is effective at improving disease in a dose dependent manner. See FIG. 8.

### Example 7: In Vivo Asthma Model

Efficacy of Compound 1 was assessed on an experimental ovalbumin (OVA)-induced acute asthma model. The OVA model is a widely used pre-clinical allergic asthma model and recapitulates many of the hallmarks of allergic asthma in humans. These include elevated IgE and TH2 related cytokines, mucus hypersecretion, airway inflammation, goblet cell hyperplasia, epithelial hypertrophy, and airway hyperreactivity to stimuli.

Mice were sensitized with an intraperitoneal (IP) injection of OVA and adjuvant on days 0 and 7. Mice were then challenged with intranasal delivery of OVA (10-200 μg) in saline on days 13-15 with endpoints conducted on day 16. Endpoints in this model generally included total and differential cell counts as well as inflammatory mediator content in the broncho-alveolar lavage fluid, airway hyperreactivity and detailed lung mechanics measured with the flexiVent™ rodent mechanical ventilator as well as histopathology and immunohistochemistry on lung sections.

| | | | | | | OVA | |
|---|---|---|---|---|---|---|---|
| | | OVA | | | | Challenge | |
| | | Sensitization | | | Route & | (IN) Day | Terminal |
| | No. | (IP) Day 0 and | | | Dose | 13, 14, & | Collections |
| Group | Animals | Day 7 | Treatment | Dose | Schedule | 15 | Day 16 |
| 1 | 9 | — | — | — | — | — | 1. Blood (serum) |
| 2 | 12 | 20 μg IVA + | Vehicle | — | PO, QD | 20 μg OVA | 2. Methacholine |
| 3 | 12 | 1.5 mg Alum | Compound 1 | 1 mg/kg | Day | | challenge and |
| 4 | 12 | | | 0.1 mg/kg | 12-15 | | detailed lung |
| 5 | 12 | | Dexamethasone | 5 mg/kg | IP, QD | | mechanics |
| | | | | | Day | | 3. Lungs |
| | | | | | 12-15 | | Right lung |
| | | | | | | | BAL fluid: |
| | | | | | | | Differential |
| | | | | | | | counts |
| | | | | | | | Right lung |
| | | | | | | | tissue: frozen |
| | | | | | | | Left lung: |
| | | | | | | | fixed |
| | | | | | | | histopath |
| | | | | | | | (H&E, PAS) |

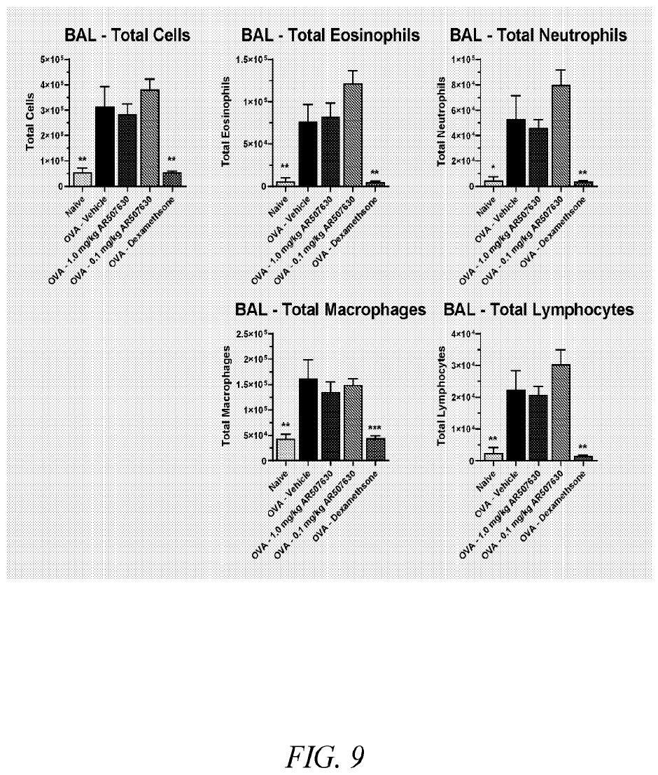

1 mg/kg and 0.1 mg/kg Compound 1 did not have significant effect on immune cells in bronchoalveolar lavage (BAL). BAL is a surrogate measure of local inflammation which is used as a readout for efficacy in this model. See FIG. 9.

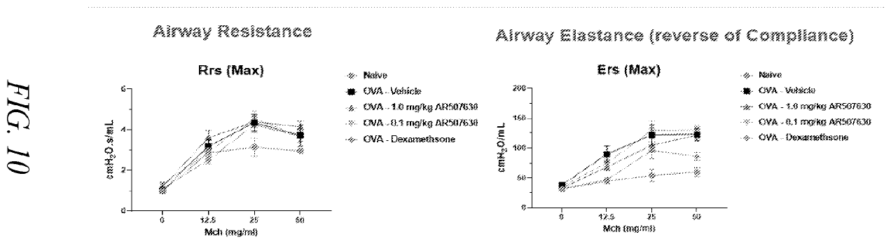

Compound 1 had no significant effect on lung function measured by airway resistance or elastance. Airway hypersensitivity response (AHR) is a surrogate measure of lung function, which is used as a measure of overall efficacy in this model. This data suggests that 1 mg/kg Compound 1 has no effect improving disease via lung compliance. See FIG. 10.

Other uses of the disclosed methods will become apparent to those in the art based upon, inter alia, a review of this patent document.

1. A method of treating an individual with atopic dermatitis comprising: administering to the individual in need thereof a pharmaceutical dosage form comprising a therapeutically effective amount of 1-{2-fluoro-4-[5-(4-isobutylphenyl)-1,2,4-oxadiazole-3-yl]benzyl}-3-azetidinecarboxylic acid (Compound 1), or a pharmaceutically acceptable salt, solvate, or hydrate thereof.

2. The method of claim 1, wherein the atopic dermatitis is chronic atopic dermatitis.

3. The method of claim 1, wherein the atopic dermatitis is moderate to severe atopic dermatitis.

4. (canceled)

5. A method of treating an individual with an eosinophilic GI disease, comprising: administering to the individual in need thereof a pharmaceutical dosage form comprising a therapeutically effective amount of 1-{2-fluoro-4-[5-(4-isobutylphenyl)-1,2,4-oxadiazole-3-yl]benzyl}-3-azetidinecarboxylic acid (Compound 1), or a pharmaceutically acceptable salt, solvate, or hydrate thereof.

6. The method of claim 5, wherein the eosinophilic GI disease is selected from eosinophilic esophagitis (EoE), eosinophilic gastritis (EG), eosinophilic gastroenteritis (EGE), and eosinophilic colitis (EC).

7. The method of claim 5, wherein the eosinophilic GI disease is eosinophilic esophagitis (EoE).

8. A method of treating an individual with asthma, comprising: administering to the individual in need thereof a pharmaceutical dosage form comprising a therapeutically effective amount of 1-{2-fluoro-4-[5-(4-isobutylphenyl)-1,2,4-oxadiazole-3-yl]benzyl}-3-azetidinecarboxylic acid (Compound 1), or a pharmaceutically acceptable salt, solvate, or hydrate thereof.

9. The method of claim 8, wherein asthma is chosen from bronchial asthma, allergic asthma, intrinsic asthma, extrinsic asthma, dust asthma, chronic or inveterate asthma, late asthma and airway hyper-responsiveness.

10. The method of claim 8, wherein asthma is chosen from exercise-induced asthma, occupational asthma, and allergy-induced asthma.

11. The method of claim 1, wherein the therapeutically effective amount is equivalent to about 0.1 mg to 2.5 mg of Compound 1.

12. The method of claim 11, wherein the therapeutically effective amount is equivalent to about 0.15 mg of Compound 1.

13. The method of claim 11, wherein the therapeutically effective amount is equivalent to about 0.25 mg of Compound 1.

14. The method of claim 11, wherein the therapeutically effective amount is equivalent to about 2.5 mg of Compound 1.

15. The method of claim 1, wherein Compound 1, or a pharmaceutically acceptable salt, solvate, or hydrate thereof, is administered once daily.

16. The method of claim 1, wherein the Compound 1, or a pharmaceutically acceptable salt, hydrate, or solvate thereof, is administered orally.

17. The method of claim 1, wherein the Compound 1, or a pharmaceutically acceptable salt, hydrate, or solvate thereof, is formulated as a capsule or tablet suitable for oral administration.
